(19) United States
(12) Patent Application Publication
(10) Pub. No.: US 2023/0190669 A1
(43) Pub. Date: Jun. 22, 2023

(54) DRUG DELIVERY DEVICE INCLUDING PATCH SEGMENTS

(71) Applicant: KINDEVA DRUG DELIVERY L.P., Woodbury, MN (US)

(72) Inventors: Matthew Tyler Zabel, Lake Elmo, MN (US); Philip Stankard, Woodbury, MN (US); Ethan Trepp, Minneapolis, MN (US); Timothy A. Peterson, Forest Lake, MN (US); Keith Dahmen, North St. Paul, MN (US)

(73) Assignee: KINDEVA DRUG DELIVERY L.P., Woodbury, MN (US)

(21) Appl. No.: 18/014,444

(22) PCT Filed: Jul. 6, 2021
(86) PCT No.: PCT/US2021/040434

§ 371 Date: Jan. 4, 2023

Related U.S. Application Data
(60) Provisional application No. 63/048,487, filed on Jul. 6, 2020.

(51) Int. Cl. A61K 9/70; A61K 31/573

(52) U.S. Cl. CPC A61K 9/7092; A61K 31/573; A61K 9/7069

(57) ABSTRACT

Various embodiments of a drug delivery device and a method of using such device are disclosed. The drug delivery device includes a backing layer having a first major surface and a second major surface, and an array of patch segments defined by segment separation lines disposed in the backing layer. Each segment separation line is adapted to enhance separation of one or more patch segments from the array of patch segments. Further, a patch segment of the array of patch segments includes a polygonal shape.

### CROSS-REFERENCE TO RELATED APPLICATIONS

This application claims the benefit of U.S. Patent Application Ser. No. 63/048,487, filed Jul. 6, 2020, and is related to co-filed U.S. Patent Application Ser. No. 63/048,516, (Attorney Docket No. 83037US002), entitled DRUG DELIVERY DEVICE FOR DELIVERY OF CLOBETASOL PROPIONATE, the disclosures of which are incorporated by reference herein in their entireties.

### BACKGROUND

Drug delivery devices such as transdermal and topical patches and bandages typically include a backing layer, an adhesive layer, and a release liner disposed such that the adhesive layer is between the backing layer and the release liner. Such devices can also include one or more active pharmaceutical ingredients disposed on or within the adhesive layer that can provide various therapeutic benefits to a user that applies such devices to the user's skin. These active pharmaceutical ingredients can include compounds such as clobetasol propionate, which is a corticosteroid typically used topically for treatment of atopic dermatitis, psoriasis, and other skin conditions. Clobetasol propionate is currently available by prescription in a variety of topical dosage forms, including ointments, lotions, creams, and foams.

### SUMMARY

In general, the present disclosure provides various embodiments of a drug delivery device that includes an array of patch segments that are defined by one or more segment separation lines disposed in a backing layer of the device. One or more of these patch segments can be separated from the array of patch segments along the one or more segment separation lines. This configuration of removable patch segments can allow a user to tailor the drug delivery device to cover a selected area or areas of the user's skin. Further, the separated patch segments can also be applied to the user's skin to cover additional portions or areas.

In one aspect, the present disclosure provides a drug delivery device that includes a backing layer having a first major surface and a second major surface, and an array of patch segments defined by segment separation lines disposed in the backing layer. Each segment separation line is adapted to enhance separation of one or more patch segments from the array of patch segments. Further, a patch segment of the array of patch segments includes a polygonal shape having at least five sides and no greater than seven sides.

In another aspect, the present disclosure provides a web that includes a backing layer having a first major surface and a second major surface, and drug delivery devices defined by patch separation lines disposed in the backing layer. Each patch separation line is adapted to enhance separation of one or more drug delivery devices from the web. Further, each drug delivery device includes an array of patch segments defined by segment separation lines disposed in the backing layer, where each segment separation line is adapted to enhance separation of one or more patch segments from the array of hexagonal patch segments of each patch. In addition, a patch segment of the array of patch segments includes a polygonal shape having at least five sides and no greater than seven sides.

In another aspect, the present disclosure provides a method that includes removing a release liner from a drug delivery device, where the drug delivery device includes a backing layer having a first major surface and a second major surface, and an array of patch segments defined by segment separation lines disposed in the backing layer. Each segment separation line is adapted to enhance separation of one or more patch segments from the array of patch segments. Further, a patch segment of the array of patch segments includes a polygonal shape having at least five sides and no greater than seven sides. The method further includes disposing the drug delivery device onto a portion of skin of a user such that an adhesive composition that is disposed on the second major surface of the backing layer of the drug delivery device is in contact with the skin of the user; separating a patch segment from the array of patch segments of the drug delivery device along a segment separation line; and removing the separated patch segment from the skin of the user.

In another aspect, the present disclosure provides a method that includes separating a patch segment from an array of patch segments of a drug delivery device along a segment separation line that is disposed in a backing layer of the drug delivery device, where the backing layer includes a first major surface and a second major surface, and further where a patch segment of the array of patch segments includes a polygonal shape having at least five sides and no greater than seven sides. The method further includes removing a release liner from the drug delivery device, and disposing the drug delivery device onto a portion of skin of a user such that an adhesive composition that is disposed on the second major surface of the backing layer of the drug delivery device is in contact with the skin of the user.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

Throughout the specification, reference is made to the appended drawings, where like reference numerals designate like elements, and wherein:

FIG. 1 is a schematic plan view of one embodiment of a drug delivery device.

FIG. 2 is a schematic cross-section view of a portion of the drug delivery device of FIG. 1.

FIG. 3 is a schematic plan view of the drug delivery device with one or more patch segments separated from an array of patch segments of the device.

FIG. 4 is a schematic perspective view of portions of the drug delivery device of FIG. 1 disposed on skin of a hand of a user.

FIG. 5 is a schematic plan view of a portion of another embodiment of a drug delivery device.

FIG. 6 is a schematic plan view of a portion of another embodiment of a drug delivery device.

FIG. 7 is a schematic plan view of a portion of another embodiment of a drug delivery device.

FIG. 8 is a schematic plan view of a web that includes one or more drug delivery devices.

FIG. 9 is a flowchart of one embodiment of a method of utilizing the drug delivery device of FIG. 1.

FIG. 10 is a flowchart of another embodiment of a method of utilizing the drug delivery device of FIG. 1.

### DETAILED DESCRIPTION

In general, the present disclosure provides various embodiments of a drug delivery device that includes an array of patch segments that are defined by one or more segment separation lines disposed in a backing layer of the device. One or more of these patch segments can be separated from the array of patch segments along the one or more segment separation lines. This configuration of removable patch segments can allow a user to tailor the drug delivery device to cover a selected area or areas of the user's skin. Further, the separated patch segments can also be applied to the user's skin to cover additional portions or areas. The various embodiments of drug delivery devices described herein can be utilized in any suitable application, e.g., transdermal or topical drug delivery, medical securement tapes that are customizable in size to contour, e.g., around IV tubing and monitoring leads, and medical wound dressings.

Drug delivery devices such as transdermal and topical patches can be adapted to provide active pharmaceutical ingredients to a user. For example, a topical patch can be adapted to provide one or more active pharmaceutical ingredients to skin of a user to treat such conditions as psoriasis and dermatitis. Such skin conditions can, however, generate treatment areas such as lesions that vary in size and location on the human anatomy. Further, known topical patches are not easily customizable such that the patches cover only the lesions while not occluding healthy skin surrounding such lesions. Portions of these known topical patches that are not utilized to treat lesions may be wasted and cannot be reapplied. Also, such topical patches can be difficult to handle given their size and the lack of rigidity of a conformable backing layer of the patch. These conformable backing layers are not, however, typically conformable on or around anatomical joints as the size of the patch prohibits the layer from remaining in contact with the skin of the user as the patch bends.

One or more embodiments of drug delivery devices described herein can provide various advantages over currently available transdermal or topical patches. For example, one or more embodiments of devices described herein can include an array of patch segments, where one or more of these patch segments can be removed from the device along one or more segment separation lines such that the device is tailored to cover one or more lesions on the skin of the user. Because of this ability to tailor the size and shape of the device, numerous configurations and sizes of lesions can be treated without occluding a substantial portion of skin that surrounds the lesions. Further, the one or more patch segments that are removed from the device can be disposed on other lesions or stored and used at a later time as the device can include a liner that can remain intact on these unused segments. Further, in one or more embodiments, the array of patch segments can be closely packed such that one or more sides or edges of a patch segment are nested with one or more sides or edges of adjacent patch segments, thereby eliminating trim or waste material that in typical patches may be disposed between segments. Because of the nested, closely-packed arrangement of the array of patch segments, one or more embodiments of drug delivery devices described herein can be formed using high-yield manufacturing processes that can reduce trim or waste. The one or more segment separation lines that define the closely-packed array of patch segments can allow the user to tailor the shape of the device to fit a particular size of lesion or other treatment site on the skin. These segment separation lines can also provide additional flexibility to the device such that the device remains in contact with the skin of the user when the device is disposed on a nonplanar portion of the skin or over a joint or other articulating surface of the user's body. Further, a patch can be cleanly removed from the skin while gels or creams may leave residue even after being wiped off with a cloth or tissue.

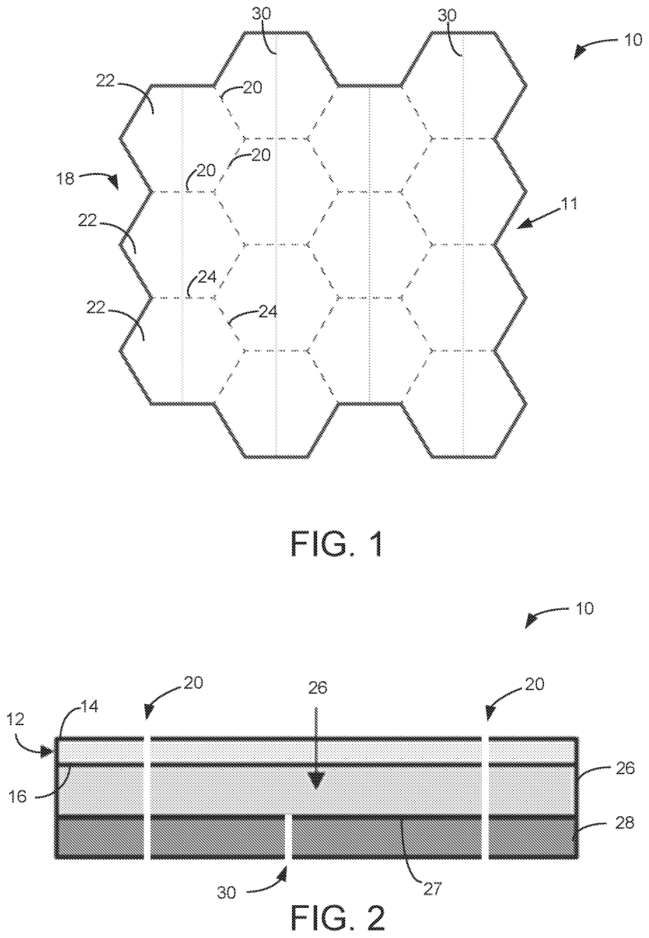

FIGS. 1-4 are various views of one embodiment of a drug delivery device 10. The device 10 includes a backing layer 12 that has a first major surface 14 and a second major surface 16, and an array 18 of patch segments 22. The array 18 is defined by one or more segment separation lines 20 that are disposed in the backing layer 12. Each segment separation line 20 is adapted to enhance separation of one or more patch segments 22 from the array 18 of patch segments 22. Further, each patch segment 22 of the array 18 can include any suitable shape or shapes, e.g., at least one of elliptical, rectilinear, triangular, or irregular shapes. In one or more embodiments, one or more of the patch segments 22 can take a polygonal shape. In one or more embodiments, such polygonal shape can include any suitable number of sides or edges 24, e.g., three, four, five, six, seven, or more sides. In one or more embodiments, one or more patch segments 22 can take a polygonal shape that includes at least five sides 24 and no greater than seven sides.

As shown in FIG. 2, the device 10 can also include an adhesive composition 26 disposed on the second major surface 16 of the backing layer 12, and one or more active pharmaceutical ingredients (not shown) disposed on or within the adhesive composition. Further, the device 10 can also include a release liner 28 disposed such that the adhesive composition 26 and the one or more active pharmaceutical ingredients are disposed between the backing layer 12 and the release liner. Removal of the release liner 28 exposes a skin contact surface 27 of the adhesive composition 26.

In general, the device 10 can take any suitable shape and have any suitable dimensions. For example, as shown in FIG. 1, the device 10 includes a perimeter 11 that can take any suitable shape. The perimeter 11 is defined by sides 24 of the patch segments 22. In one or more embodiments, the array 18 of the device 10 can be disposed on a carrier having edges that would then define the perimeter 11 of the device 10. As further described herein, one or more patch segments 22 can be removed from the device 10, thereby altering the perimeter 11 of the device to tailor the overall shape and dimensions of the device. The perimeter 11 defines an area of the device 10. The device 10 can have any suitable area. In one or more embodiments, the device 10 can have an area of at least 20 cm² and no greater than 200 cm². In one or more embodiments, the device 10 can have an area of at least 50 cm² and no greater than 150 cm².

The device 10 can be utilized for any suitable application to provide one or more active pharmaceutical ingredients to the user. In one or more embodiments, the device 10 can include a transdermal patch as described, e.g., in U.S. Pat. No. 9,375,510 to DiZio et al. and entitled TRANSDERMAL ADHESIVE COMPOSITIONS, DEVICES AND METHODS. Further, in one or more embodiments, the device 10 can include a topical patch, e.g., patch that is disposed on a surface of the user's skin and provides one or more active pharmaceutical ingredients topically to the skin as described in U.S. Pat. No. 6,096,334 to Rolf et al. and entitled ADHESIVE PATCH FOR APPLYING MEDICATION TO THE SKIN AND METHOD.

Further, the drug delivery device 10 can be disposed individually or in roll form in any suitable pouch for storage, e.g., a foil lined pouch. In one or more embodiments, the drug delivery device 10 can be provided in a rolled or stacked form suitable for use with a dispensing apparatus.

The backing layer 12 of the device 10 can include any suitable materials. Suitable materials include conventional flexible backing materials used for pressure sensitive adhesive tapes, including, for example, polyethylene (particularly low density polyethylene, linear low density polyethylene, metallocene polyethylenes, or high density polyethylene), polypropylene, polyesters such as polyethylene terephthalate, randomly oriented nylon fibers, ethylene-vinyl acetate copolymer, polyurethane, natural fibers such as rayon and the like. Suitable materials also include materials that are layered, such as polyethylene terephthalate-aluminum-polyethylene composites. In one or more embodiments, the backing layer 12 may be formed from low density polyethylene (LDPE) film, which is a low modulus film relative to other transdermal backings. One exemplary low density polyethylene film is a 1.7 mil LDPE film (COTRAN, 3M Corp., St. Paul, Minn.). Further, the backing layer 12 can be treated using any suitable technique or techniques, e.g., the techniques described in co-filed U.S. Patent Application Ser. No. 63/048,516 (Attorney Docket No. 83037US002), entitled DRUG DELIVERY DEVICE FOR DELIVERY OF CLOBETASOL PROPIONATE.

The backing layer 12 can further include indicia or printed images disposed on or in the layer. For example, a decorative design can be disposed on at least one of the first or second major surfaces 14, 16 or within the backing layer 12. Further, a separate layer that includes indicia or printed images can be disposed on the backing layer 12 using any suitable technique. In one or more embodiments, indicia can be disposed on or within the backing layer 12 to indicate to the user the location of one or more of the segment separations lines 20 to facilitate separation of one or more patch segments 22 from the array 18.

Disposed in the backing layer 12 is the array 18 of patch segments 22. The array 18 can include any suitable number of patch segments 18. Further, the array 18 is defined by segment separation lines 20 that are disposed in the backing layer 12 using any suitable technique or techniques. Each segment separation line 20 is adapted to enhance separation of one or more patch segments 22 from the array 18. The device 10 can include any suitable number of segment separation lines 20. Further, each segment separation line 20 can take any suitable shape, e.g., straight, curved, etc. Further, each separation line 20 can intersect one or more additional separation lines in any suitable pattern or patterns.

The segment separation lines 20 are disposed in the backing layer 12 and can extend through the backing layer. In one or more embodiments, one or more of the segment separation lines 20 can extend through the adhesive composition 26 as shown in FIG. 2. Further, in one or more embodiments, one or more segment separation lines 20 can extend through the backing layer 12, the adhesive composition 26, the one or more active pharmaceutical ingredients, and the release liner 28.

Each segment separation line 20 can be formed using any suitable technique or techniques. In one or more embodiments, one or more of the segment separation lines 20 can include one or more perforations formed using any suitable technique. Each perforation can include any suitable dimensions. In one or more embodiments, one or more of the perforations can include a length of at least 0.25 mm and no greater than 1.5 mm. Further, the perforations can have any suitable width. Segment separation lines 20 that include perforations can also include one or more tie portions disposed between the perforations. The tie portions can have any suitable dimensions, e.g., a length of at least 0.125 mm and no greater than 1 mm. Further, in one or more embodiments, one or more segment separation lines 20 can include one or more thinned portions to provide one or more lines of weakness along which patch segments 22 can be separated from the array 18. Any suitable technique can be utilized to provide these thin portions.

As stated herein, the one or more segment separation lines 20 define the array 18 of patch segments 22. Each segment 22 can take any suitable shape and have any suitable dimensions. In one or more embodiments, one or more of the patch segments 22 can include a polygonal shape. As used herein, the term “polygonal” refers to a planar shape having at least three straight sides and angles. The polygonal shapes of the one or more segments 22 can have any suitable number of sides. Polygonal patch segments 22 can take the shape of a regular polygon or an irregular polygon. As used herein, the term “regular polygon” refers to a polygon that is equal angular (i.e., all angles are equal) and equilateral (i.e., all sides have the same length). Further, as used herein, the term “irregular polygon” is any polygon that is not a regular polygon.

In one or more embodiments, one or more of the patch segments 22 can take a polygonal shape having at least five sides and no greater than seven sides. Further, in one or more embodiments, one or more patch segments 22 can take a hexagonal shape to provide one or more hexagonal patch segments. In one or more embodiments, one or more patch segments 22 can take a regular hexagonal shape. In one or more embodiments, one or more of the patch segments 22 can take an irregular hexagonal shape.

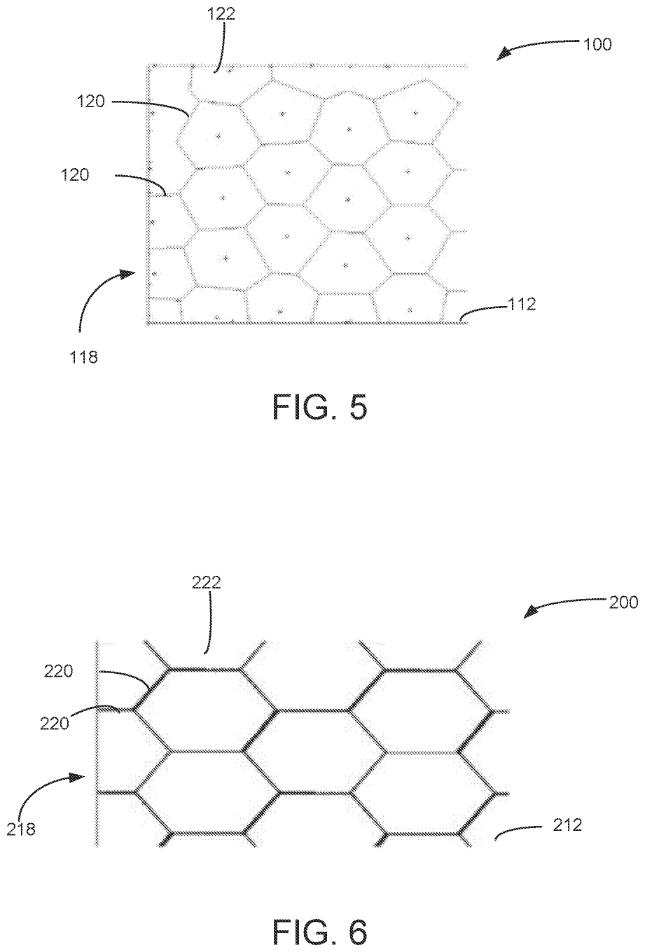

All of the patch segments 22 of the array 18 can have the same planar shape, e.g., the array has regular hexagonal patch segments. In one or more embodiments, one or more patch segments 22 of the array 18 can take a shape that is different from a shape of another patch segment. For example, FIG. 5 is a schematic plan view of a portion of another embodiment of a drug delivery device 100. All of the design considerations and possibilities regarding the drug delivery device 10 of FIGS. 1-4 apply equally to the device 100 of FIG. 5. The device 100 includes an array 118 of patch segments 122 defined by segment separation lines 120 disposed in a backing layer 112. One difference between device 100 of FIG. 5 and device 10 of FIGS. 1-4 is that device the patch segments 122 of the array 118 each take an irregular hexagonal shape. Even though the array 118 includes irregular hexagonal-shaped patch segments 122, such segments are closely-packed such that little or no space is disposed between adjacent patch segments 122. In one or more embodiments, the entire surface area of the backing layer 112 is filled by polygonal, e.g., hexagonal patch segments 122.

Further, for example FIG. 6 is a schematic plan view of a portion of another embodiment of a drug delivery device 200. All of the design considerations and possibilities regarding the drug delivery device 10 of FIGS. 1-4 apply equally to the drug delivery device 200 of FIG. 6. As shown in FIG. 6, the device 200 includes an array 218 of patch segments 222 defined by separation lines 220 disposed in a backing layer 212. The array 218 includes one or more patch segments 222. As shown in FIG. 6, each patch segment 222 takes an irregular hexagonal shape.

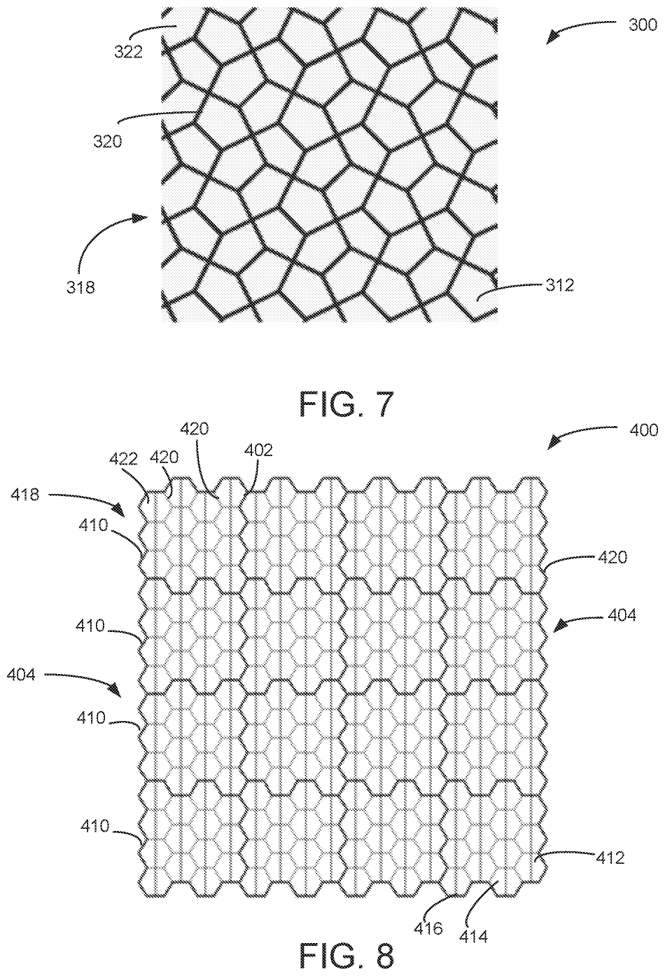

As mentioned herein, each patch segment of a drug delivery device can have any suitable number of sides. For example, FIG. 7 is another embodiment of a drug delivery device 300. All of the design considerations and possibilities regarding the drug delivery device 100 of FIGS. 1-4 apply equally to the drug delivery device 300 of FIG. 7. The device 300 includes an array 318 of patch segments 322 that are defined by one or more separation lines 320 disposed in a backing layer 312. As shown in FIG. 7, each patch segment 322 of the array 318 takes a five-sided polygonal shape, i.e., a pentagonal shape. Each pentagonal shape is irregular. In one or more embodiments, one or more of the patch segments 322 can take a regular pentagonal shape.

Returning to FIGS. 1-4, each patch segment 22 can have any suitable area in a plane of the first major surface 14 of the backing layer 12. In one or more embodiments, at least one patch segment 22 of the array 18 has an area of at least 50 mm² and no greater than 2000 mm². Each of the patch segments 22 can have the same area. In one or more embodiments, one or more patch segments 22 can have an area that is different from one or more additional patch segments.

As mentioned herein, the patch segments 22 can be closely-packed in the array 18 such that little or no backing layer material is disposed between adjacent segments. In one or more embodiments, one or more of the patch segments 22 can be nested with adjacent patch segments such that one or more sides 24 of the patch segment is shared with one or more sides of adjacent patch segments. In one or more embodiments, an area of the backing layer 12 of the device 10 can be equal to an area of the array 18 of patch segments 22 such that little or no backing layer material is disposed between patch segments.

As mentioned herein, the device 10 can also include the adhesive composition 26 disposed on the second major surface 16 of the backing layer 12. The adhesive composition 26 can include any suitable materials. In one or more embodiments, the adhesive composition can include any polymer, or combination of polymers, that provides a desirable combination of adhesion to the skin and delivery of the one or more active pharmaceutical ingredients. Exemplary suitable polymers include, but are not limited to, acrylates, silicones, polyisobutylenes, and mixtures thereof. In one or more embodiments, the adhesive composition can include a silicone adhesive composition. Other embodiments of suitable adhesive compositions are described, e.g., in U.S. Patent Application Ser. No. 63/048,516, (Attorney Docket No. 83037US002), entitled DRUG DELIVERY DEVICE FOR DELIVERY OF CLOBETASOL PROPIONATE.

In one or more embodiments, the device 10 can also include one or more active pharmaceutical ingredients disposed on or within the adhesive composition 26. Such pharmaceutical ingredients can be disposed in any suitable location within the device 10. In one or more embodiments, the one or more active pharmaceutical ingredients can be disposed within the adhesive composition 26. In one or more embodiments, the one or more active pharmaceutical ingredients can be disposed between the adhesive composition 26 and the second major surface 16 of the backing layer 12. Further, in one or more embodiments, the one or more active pharmaceutical ingredients can be disposed between the adhesive composition 26 and the release liner 28. In such embodiment, the one or more active pharmaceutical ingredients can form one or more portions of the skin contacting surface 27 of the device 10.

The one or more active pharmaceutical ingredients can include any suitable composition or compositions. For example, suitable compositions include buprenorphine, clonidine, estradiol, ethinyl estradiol, fentanyl, flurandrenolide, granisetron, lidocaine, menthol, methyl salicylate, methylphenidate, nicotine, nitroglycerine, norelgestromin, oxybutynin, rivastigmine, scopolamine, selegiline, and testosterone, which are commercially available in the form of transdermal or topical devices. Other examples include anti-inflammatory compounds, both steroidal (e.g., clobetasol propionate, hydrocortisone, prednisolone, triamcinolone) and nonsteroidal (e.g., naproxen, piroxicam, diclofenac); bacteriostatic agents (e.g., chlorhexidine, hexylresorcinol); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotazoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors (e.g., A64077), and angiotensin converting enzyme inhibitors (e.g., captopril, lisinopril); other antihypertensives (e.g., propranolol); leukotriene antagonists (e.g., ICI204,219); anti-ulceratives such as H2 antagonists; steroidal hormones (e.g., progesterone); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinolin-4-amine, N-[4-(4-amino-2-ethyl-1H-imidazo[4,5-c]quinolin-1-yl)butyl]methanesulfonamide, and acyclovir); local anesthetics (e.g., benzocaine, propofol, tetracaine, prilocaine); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, hydromorphone hydrochloride); peptide hormones (e.g., human or animal growth hormones, LHRH, parathyroid hormones); cardioactive products such as atriopeptides; antidiabetic agents (e.g., insulin, exanatide); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin, enoxaparin sodium); analgesics (e.g., acetaminophen); antimigraine agents (e.g., ergotamine, melatonin, sumatripan, zolmitriptan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; dopamine agonists (e.g., apomorphine); GnRH agonists (e.g., leuprolide, goserelin, nafarelin); fertility hormones (e.g., hCG, hMG, urofollitropin); interferons (e.g., interferon-alpha, interferon-beta, pegylated interferon-alpha); and the like, as well as pharmaceutically acceptable salts and esters thereof. Additional suitable pharmaceutical ingredients and concentrations, and methods of making such ingredients are further described in U.S. Patent Application Ser. No. 63/048,516, (Attorney Docket No. 83037US002), entitled DRUG DELIVERY DEVICE FOR DELIVERY OF CLOBETASOL PROPIONATE.

As described herein, the device 10 can also include the release liner 28. The release liner 28 can be disposed such that the adhesive composition 26 and the one or more active pharmaceutical ingredients are disposed between the backing layer 12 and the release liner 28. The release liner 28 can include any suitable release liner, e.g., conventional release liners that include a known sheet material such as a polyester web, a polyethylene web, poly propylene web, or a polyethylene coated paper coated web with a suitable floor polymer or silicone based coating. Suitable release liners are further described, e.g., in U.S. Patent Application Ser. No. 63/048,516, (Attorney Docket No. 83037US002), entitled DRUG DELIVERY DEVICE FOR DELIVERY OF CLOBETASOL PROPIONATE.

The release liner 28 can also include one or more slits 30 disposed in the liner as shown in FIGS. 1-2. The slits 30 can be disposed in the release liner 28 using an any suitable techniques or techniques. Further, the slits 30 can take any suitable shape and have any suitable dimensions. In one or more embodiments, the slits 30 can take a sinusoidal shape. Further, in one or more embodiments, the slits 30 can include two or more slit portions separated by a tie portion. The slits portions and the tie portions can have any suitable dimensions. In one or more embodiments, a slit 30 can be disposed in the release liner 28 along each row of patch segments 22 as is shown in FIG. 1.

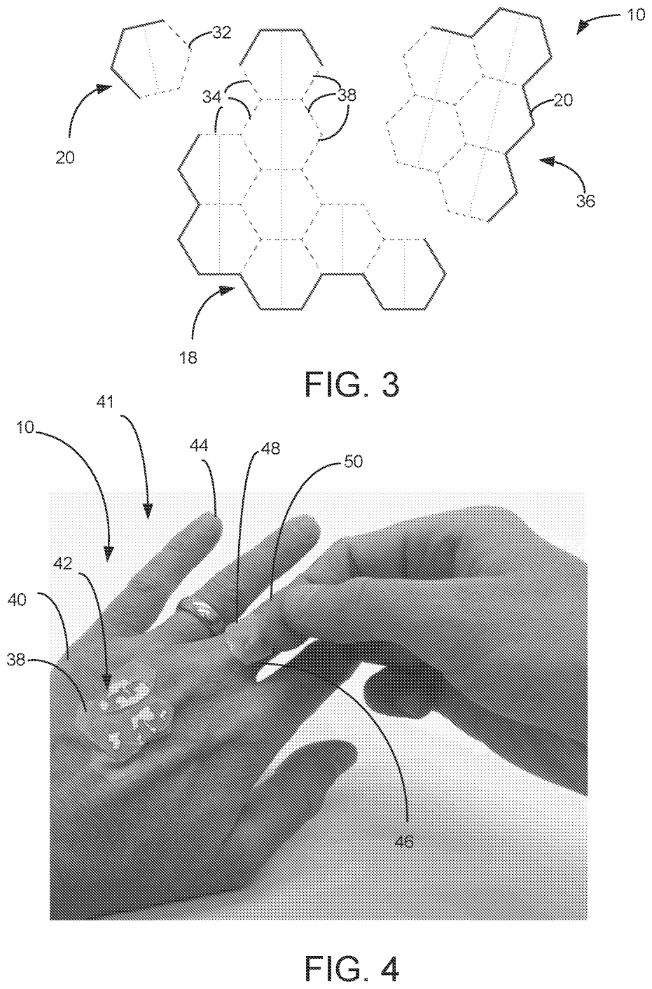

As mentioned herein, one or more patch segments 22 can be removed from the array 18 along one or more segment separation lines 20. For example, as shown in FIG. 3, a patch segment 32 is separated from the array 18 of drug delivery device 10 along separation lines 34. Further, a portion 36 of the array 18 is also separated from the array along separation lines 38. The portion 36 can include any suitable number of patch segments 20 and can take any suitable shape or shapes. In one or more embodiments, the patch segment 32 and the portion 36 are removed from the array 18 prior to the release liner 28 being removed from the adhesive composition 26 such that portions of the release liner remain intact with the patch segment and the portion. As a result, one or both of the patch segment 32 and the portion 36 can be retained and used at a later time or disposed on other portions of the skin of the user by removing the release liner and applying the patch segment or the portion to the skin such that the skin contacting surface 27 of the adhesive composition 26 is in contact with the skin and retains the segment or portion on the skin.

For example, as shown in FIG. 4, a first portion 38 of the drug delivery device 10 is disposed on skin 40 of the user located on a back 42 of a hand 44 of the user. Further, a second portion 46 of the device 10 is disposed on the skin 40 of the user on a back surface 48 of a finger 50 of the user. As is further described herein, portion or portions of the device 10 can be disposed on the skin using any suitable technique. For example, the first portion 38 and the second portion 46 can be separated from the array 18 of the device 10, and portions of the release liner 28 corresponding with the first and second portions of the device 10 can be removed. Once the liner 28 is removed, the first and second portions 38, 46 can be disposed on the skin 40 of the user by pressing the portions against the skin such that the adhesive composition 26 adheres to the skin in the desired locations. Once disposed on the skin 40, the portions 38, 46 of the device 10 can provide the one or more active pharmaceutical ingredients to the user's skin.

The various embodiments of drug delivery devices described herein can be manufactured using any suitable technique or techniques. For example, FIG. 8 is a schematic plan view of one embodiment of a web 400. The web 400 includes a backing layer 412 that has a first major surface 414 and a second major surface 416. The web 400 also includes one or more drug delivery devices 410 defined by patch separation lines 402 that are disposed in the backing layer 412. Each patch separation line 402 is adapted to enhance separation of one or more drug delivery devices 410 from the web 400. Each drug delivery device 410 can include any suitable drug delivery device, e.g., drug delivery device 10 in FIG. 1-4. All of the design considerations and possibilities regarding the drug delivery device 10 of FIG. 1-4 apply equally to drug delivery devices 410 of FIG. 8. For example, one or more of the drug delivery devices 410 can include an array of patch segments defined by segment separation lines disposed in the backing layer, where each segment separation line is adapted to enhance separation of one or more patch segments from the array of hexagonal patch segments of each patch. Further, the web 400 can also include an adhesive composition disposed on the second major surface 416 of the backing layer 412, and one or more active pharmaceutical ingredients disposed on or within the adhesive composition as is described regarding drug delivery device 10 of FIGS. 1-4.

The web 400 can also include a release liner (e.g., release liner 28 of FIGS. 1-4) that is disposed such that the adhesive composition and the one or more active pharmaceutical ingredients are disposed between the backing layer 412 and the release liner. The release liner can include one or more slits (e.g. slit 30 of FIG. 2) disposed in the release liner that can take any suitable shape or shapes and have any suitable dimensions. Further, in one or more embodiments, the patch separation lines 402 can extend through the backing layer 412, the adhesive composition, the one or more active pharmaceutical ingredients, and the release liner such that one or more of the devices 410 can be separated from the web 400 along such patch separation lines. Further, the web 400 can also include segment separation lines 420 disposed in the backing layer 412 that are adapted to enhance separation of one or more patch segments 422 from an array 418 of patch segments as is described in regard to device 10 of FIGS. 1-4.

In one or more embodiments, the drug delivery devices 410 of the web 400 and the patch segments 422 of each device can be nested or closely packed such that little or no material of the backing layer 412 is disposed between devices or segments within such devices. Further, the web 400 can be formed such that an edge 404 or edges of the web 400 parallel to a machine direction of the web (in a direction towards the top of FIG. 8) is defined by segment separation lines 420 of the devices 410. Such configuration can reduce the amount of trim or waste formed during manufacture of the web 400.

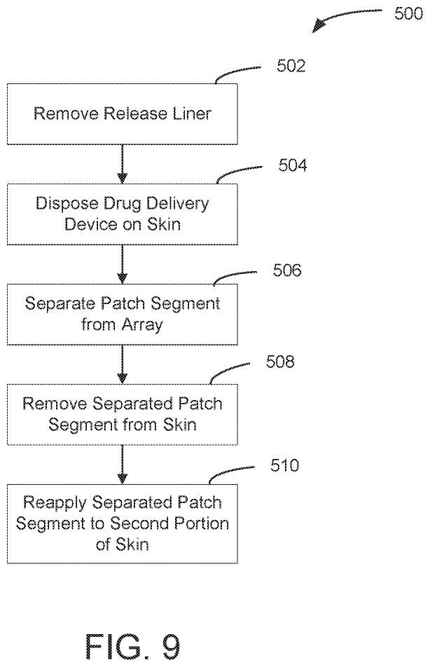

The various embodiments of drug delivery devices described herein can be utilized using any suitable technique or techniques. For example, FIG. 9 is a flowchart of one embodiment of a method 500 of utilizing drug delivery device 10. Although described regarding drug delivery device 10 of FIGS. 1-4, the method 500 can be utilized with any drug delivery device. At 502, the release liner 28 can be removed from the drug delivery device 10. Any suitable technique or techniques can be utilized to remove the release liner 289. In one or more embodiments, the release liner 28 can include one or more slit 30 that can enable the user to peel the release liner from the adhesive composition 26. At 504, the drug delivery device 10 is disposed onto a portion of skin of the user (e.g., portion 42 of skin 40 of FIG. 4) such that the skin contacting surface 27 of the adhesive composition 26 is in contact with the skin of the user. Further, one or more patch segments 22 can be separated from the array 18 of patch segments along one or more segment separation lines 20 using any suitable technique or techniques at 506. For example, in one or more embodiments, the user may tear one or more patch segments 22 from the array 18 along one or more perforations that define the segment separation lines 20. At 508, the patch segment or segments 22 separated from the array 18 can be removed from the skin of the user and repositioned on one or more additional portions of the skin of the user. For example, at 510, such separated patch segment or segments 22 can be reapplied to second or additional portions of the skin (e.g., as shown in FIG. 4).

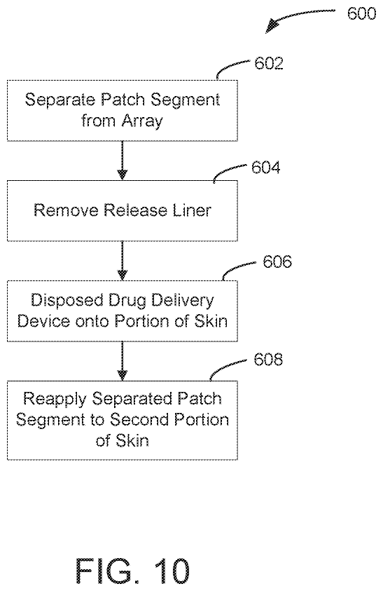

In the method 500, the array 18 is applied to the skin of the user prior to separation of one or more patch segments 22 from the array. In one or more embodiments, one or more patch segments 22 can be removed from the array 18 prior to application of the array to the skin. For example, FIG. 10 is a flowchart of another embodiment of a method 600 for utilizing the drug delivery device 10. Although described regarding the drug delivery device 10 of FIGS. 1-4, the method 600 can be utilized with any suitable drug delivery device. At 602, one or more patch segments 22 can be separated from the array of patch segments 18 of the device 10 along one or more segment separation lines 20 that are disposed in the backing layer 12 of the device using any suitable technique or techniques. Further, at 604, one or more portions of the release liner 28 can be removed from the device 10 using any suitable technique or techniques. The drug delivery device 10 can be disposed onto a portion of skin of a user such that the skin contacting surface 27 of the adhesive composition 26 is in contact with the skin of the user at 606. Further, in one or more embodiments, the separated patch segment or segments 22 can be applied to second or additional portions of the skin of the user at 608 using any suitable technique or techniques. For example, the release liner 28 of the separated patch segment or segments 22 can be removed prior to disposal of the patch segments onto the skin of the user.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1-37. (canceled)

38. A drug delivery device comprising:
a backing layer comprising a first major surface and a second major surface; and
an array of patch segments defined by segment separation lines disposed in the backing layer, wherein each segment separation line is adapted to enhance separation of one or more patch segments from the array of patch segments, and further wherein a patch segment of the array of patch segments comprises a polygonal shape comprising at least five sides and no greater than seven sides.

39. The device of claim 38, further comprising:
an adhesive composition disposed on the second major surface of the backing layer; and
one or more active pharmaceutical ingredients disposed on or within the adhesive composition.

40. The device of claim 39, further comprising a release liner disposed such that the adhesive composition and the one or more active pharmaceutical ingredients are disposed between the backing layer and the release liner.

41. The device of claim 40, wherein at least one segment separation line of the backing layer extends through the release liner.

42. The device of claim 40, further comprising a slit disposed in the release liner.

43. The device of claim 41, wherein the slit of the release liner comprises a sinusoidal shape.

44. The device of claim 39, wherein the one or more active pharmaceutical ingredients comprises clobetasol propionate.

45. The device of claim 39, wherein the adhesive composition comprises a silicone adhesive composition.

46. The device of claim 39, wherein at least one segment separation line of the backing layer extends through the backing layer, the adhesive composition, and the one or more active pharmaceutical ingredients.

47. The device of claim 38, wherein at least one patch segment of the array of patch segments comprises a hexagonal patch segment, wherein the at least one hexagonal patch segment comprises a regular hexagonal shape or an irregular hexagonal shape.

48. The device of claim 38, wherein each patch segment of the array of patch segments comprises the same shape.

49. The device of claim 38, wherein at least one patch segment of the array of patch segments comprises a shape that is different from a shape of another patch segment.

50. A web comprising:
a backing layer comprising a first major surface and a second major surface; and
drug delivery devices defined by patch separation lines disposed in the backing layer, wherein each patch separation line is adapted to enhance separation of one or more drug delivery devices from the web, wherein each drug delivery device comprises an array of patch segments defined by segment separation lines disposed in the backing layer, wherein each segment separation line is adapted to enhance separation of one or more patch segments from the array of hexagonal patch segments of each patch, and further wherein a patch segment of the array of patch segments comprises a polygonal shape comprising at least five sides and no greater than seven sides.

51. The web of claim 50, further comprising:
an adhesive composition disposed on the second major surface of the backing layer; and
one or more active pharmaceutical ingredients disposed on or within the adhesive composition.

52. The web of claim 51, further comprising a release liner disposed such that the adhesive composition and the one or more active pharmaceutical ingredients are disposed between the backing layer and the release liner.

53. The web of claim 52, further comprising a slit disposed in the release liner.

54. The web of claim 53, wherein the slit of the release liner comprises a sinusoidal shape.

55. The web of claim 51, wherein the one or more active pharmaceutical ingredients comprises clobetasol propionate.

56. A method comprising:
removing a release liner from a drug delivery device, wherein the drug delivery device comprises:
a backing layer comprising a first major surface and a second major surface; and
an array of patch segments defined by segment separation lines disposed in the backing layer, wherein each segment separation line is adapted to enhance separation of one or more patch segments from the array of patch segments, and further wherein a patch segment of the array of patch segments comprises a polygonal shape comprising at least five sides and no greater than seven sides.
disposing the drug delivery device onto a portion of skin of a user such that an adhesive composition that is disposed on the second major surface of the backing layer of the drug delivery device is in contact with the skin of the user;
separating a patch segment from the array of patch segments of the drug delivery device along a segment separation line; and
removing the separated patch segment from the skin of the user.

57. The method of claim 56, further comprising reapplying the separated patch segment to a second portion of the skin of the user.
